(19) United States
(12) Patent Application Publication
(10) Pub. No.: US 2025/0176632 A1
(43) Pub. Date: Jun. 5, 2025

(54) Aerosol Generation Device

(71) Applicant: JT International SA, Geneva (CH)

(72) Inventor: Alec Wright, Guildford (GB)

(73) Assignee: JT International SA, Geneva (CH)

(21) Appl. No.: 18/842,999

(22) PCT Filed: Mar. 8, 2023
(86) PCT No.: PCT/EP2023/055917

§ 371 Date: Aug. 30, 2024

(30) Foreign Application Priority Data
Mar. 11, 2022 (EP) 22161579.2

(51) Int. Cl. A24F 40/485; A24F 40/10; A24F 40/20; A24F 40/51; A24F 40/57

(52) U.S. Cl. CPC A24F 40/485; A24F 40/10; A24F 40/20; A24F 40/51; A24F 40/57

(57) ABSTRACT

An aerosol generation device includes: an aerosol generation chamber configured to generate an aerosol; an air flow channel arranged for drawing air through the aerosol generation chamber and for drawing aerosol from the aerosol generation chamber to an outlet; and a valve arranged in the air flow channel and configured to move between a first state and a second state, wherein air flow in the air flow channel in the first state is restricted more than air flow in the air flow channel in the second state, wherein the valve is configured to move from the first state to the second state when a pressure difference across the valve exceeds a predetermined threshold.

### FIELD OF THE INVENTION

The present disclosure relates to devices for generating an aerosol which is inhaled by a user, such as electronic cigarettes, heat-not-burn devices, and the like.

### BACKGROUND

Inhalers or aerosol generating devices such as e-cigarettes or vaping devices are becoming increasingly popular. They generally heat or warm an aerosolisable substance (such as a liquid or solid consumable) to generate an aerosol for inhalation, as opposed to burning tobacco as in conventional tobacco products. The generated aerosol may contain a flavour and/or a stimulant (e.g., nicotine or other active component). Different users of such inhalers may have different preferences about the amount and concentration of aerosol provided when they inhale from the device.

Most aerosol generating devices incorporate some form of electronic control circuit, typically including a simple computer processor, allowing a user to control operation of the aerosol generation device. However, these devices can be quite restrictive in their settings and may not offer much flexibility to the user. Even in devices that allow a user to customise settings, it requires some effort from the user and may not be intuitive.

Therefore, there exists a need for a device that provides an alternative way for a user to control properties of the aerosol to be inhaled.

### SUMMARY

According to an aspect of the present invention, there is provided an aerosol generation device comprising: an aerosol generation chamber configured to generate an aerosol; an air flow channel arranged for drawing air through the aerosol generation chamber and for drawing aerosol from the aerosol generation chamber to an outlet; and a valve arranged in the air flow channel and configured to move between a first state and a second state, wherein air flow in the air flow channel in the first state is restricted more than air flow in the air flow channel in the second state, wherein the valve is configured to move from the first state to the second state when a pressure difference across the valve exceeds a predetermined threshold.

Advantageously, the user of an aerosol generation device according to the first aspect can control the valve by inhaling more or less strongly from the outlet. For example, in some embodiments, the user can choose between performing a “light puff” or a “strong puff”. In the “light puff”, air flow is more restricted by the valve, the effective “resistance to draw” is higher, and the amount of aerosol inhaled is lower. In the “strong puff”, the air flow is less restricted by the valve after a pressure difference is initially brought above a threshold, at which point the effective “resistance to draw” becomes lower, and the amount of aerosol inhaled is higher.

Optionally, the valve is a butterfly valve. This provides a simple configuration where the valve can open and close without requiring a change to a side wall of an air flow channel.

Optionally, the air flow channel comprises a first channel in parallel with a second channel, and the valve is arranged in the second channel. In this configuration, air inhaled by a user comes from a combination of the first and second channels, and the valve can control the mixing and total flow rate of air flowing in the first and second channels. Here “parallel” means that the first channel and second channel are connected to a shared outflow end such that mixing occurs at the shared outflow end. “Parallel” does not place any limitation on the geometry of the first and second channels along their length.

Optionally, the first channel and the second channel are each connected to the aerosol generation chamber. The aerosol generation chamber is therefore a shared inflow end or a shared outflow end for the first and second channel. This can provide a variable resistance-to-draw depending upon the state of the valve, while also providing a minimum flow if the valve is closed.

As an alternative, the first channel is connected to the aerosol generation chamber and the second channel is arranged for drawing air through the aerosol generation device without passing through the aerosol generation chamber. In this configuration, the valve controls the mixing of aerosol-free air from the second channel with aerosol-enriched air from the first channel, to control a concentration of aerosol in the air inhaled by the user.

Optionally, the valve is arranged in the air flow channel upstream from the aerosol generation chamber. This can assist with decreasing the pressure in the aerosol generation chamber when the user inhales, which may increase aerosol generation in some cases. Alternatively, the valve is arranged in the air flow channel downstream from the aerosol generation chamber.

Optionally, the aerosol generation device further comprises a control circuit configured to detect or control a state of the valve. This improves the flexibility of the device. For example, by detecting the state of the valve, the device can adjust settings based on the state of the valve, for example changing a rate of aerosol generation in the aerosol generation chamber (e.g. by changing a temperature) in response to the state of the valve. On the other hand, by controlling the state of the valve, the device can, for example, control the resistance to draw of the device, or set a time limit on the less-restricted second state.

The control circuit may be configured to control the valve based on a characteristic of air flow in the air flow channel.

For example, the device may comprise a pressure sensor or flow rate sensor arranged in the air flow channel, and the control circuit may control the valve based on a pressure measurement or a flow rate measurement.

The control circuit may be configured to detect a characteristic of air flow in the air flow channel using the valve itself. More specifically, the pressure difference or flow rate may apply a force or torque to the valve, and a corresponding feedback may be induced in the control circuit. Alternatively, the control circuit may simply detect a state of the valve and infer the air flow characteristic from the valve state.

The control circuit may be configured to control the valve based on an aerosol generation parameter, such as a specified preference for aerosol concentration.

Optionally, the valve is configured to be controlled by a user. For example, in addition to being controlled based on pressure in the air flow channel, the control circuit may comprise a user interface such as a button for controlling the state of the valve.

### BRIEF DESCRIPTION OF THE DRAWINGS

FIG. 1 is a schematic cross section of an aerosol generation device according to a first embodiment;

FIG. 2 is a schematic cross section of an aerosol generation device according to a second embodiment;

FIG. 3 is a schematic cross section of an aerosol generation device according to a third embodiment;

FIG. 4 is a schematic cross section of an aerosol generation device according to a fourth embodiment.

### DETAILED DESCRIPTION

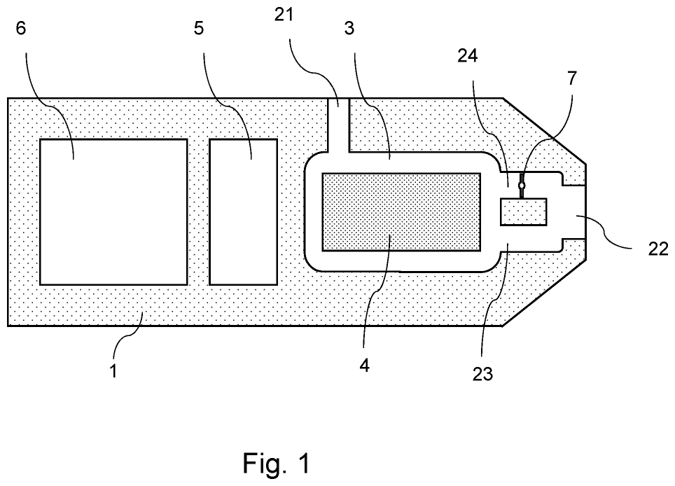

FIG. 1 is a schematic illustration of an aerosol generation device according to a first embodiment. The aerosol generation device comprises a housing 1 and an air flow channel connecting between at least one inlet 21 and an outlet 22.

The air flow channel passes through an aerosol generation chamber 3. The aerosol generation chamber 3 is configured to generate an aerosol via any known method, such as heating or burning a solid aerosol substrate 4, or vaporisation of a liquid aerosol substrate. The substrate may for example comprise tobacco and/or nicotine.

When air is drawn through the aerosol generation chamber 3 to the outlet 22, the aerosol is also drawn to the outlet 22. In many embodiments, air is drawn through the air flow channel by a user inhaling at the outlet 22. Air may instead be drawn through the air flow channel by other means such as a fan integrated in the aerosol generation device.

In this embodiment, the aerosol generation device further comprises a control circuit 5 and a power source 6, such as a battery. The control circuit 5 is configured to control the aerosol generation chamber 3 to determine when and how much aerosol is generated. For example, the control circuit 5 may control power supplied to a heating element in the aerosol generation chamber 3.

The aerosol generation device further comprises a valve 7 arranged in the air flow channel.

The valve is configured to move between different at least two different states which provide different levels of restriction for air flowing in the air flow channel. The states of the valve may include fully open, fully closed, and different degrees of partially closed.

For example, the valve may be a butterfly valve arranged across the air flow channel. When the butterfly valve is in a fully open state, its disk extends parallel to the air flow channel. When the butterfly valve is in a fully closed state, its disk extends perpendicular to the air flow channel.

In the embodiment of FIG. 1, the air flow channel is additionally split into two parallel channels 23, 24 which connect between the aerosol generation chamber 3 and the outlet 22. The valve 7 is arranged in one of the channels 24, while the other channel 23 is kept open. With this configuration, the valve 7 can be fully closed, while still allowing some restricted air flow through the other channel 23.

In an alternative embodiment, the channel 23 is omitted and the valve 7 can provide restricted air flow by partially closing. For example, a butterfly valve may be provided with a disk that is smaller than the channel 24 such that it cannot fully block the channel 24. Nevertheless, the configuration of FIG. 1 may have the advantage of reduced wear on the valve, because it is not necessary to allow air flow past the valve when it is in an air-flow-restricting state.

The valve 7 may be configured to be passively or actively controlled (or both).

If the valve 7 is configured to be passively controlled, the valve comprises a resilient structure configured to respond to a characteristic of air flow in the air flow channel 24. For example, the resilient structure may respond to a pressure difference across the valve 7 and/or an air flow rate past the valve 7.

In one case, the valve 7 may be configured with a default state of open or partially open, and may be configured to close when a user inhales strongly, in order to reduce the risk of providing a more-than-desirable quantity of aerosol.

In another case, the valve 7 may be configured with a default state of closed or partially closed, and may be configured to open when a user inhales strongly, allowing the user to subsequently inhale more vapour with reduced inhalation effort.

If the valve 7 is configured to be actively controlled, the valve comprises an actuator and the control circuit 5 (or a second separate control circuit) is configured to control the actuator.

In one case, the valve 7 may be actively controlled according to an aerosol generation parameter. For example, the state of valve 7 may be controlled in dependence upon a temperature for aerosol generation or a type of aerosol substrate (e.g. liquid or solid substrate).

In another case, a state of the valve 7 may be controlled to restrict air flow in dependence upon a rate at which aerosol is being generated in the aerosol generation chamber 3, in order to provide a consistent concentration of aerosol in the air flow.

In yet another case, the valve 7 may be actively controlled in response to a user input. For example, the control circuit 5 may include a user interface or button. An input via the user interface or button may indicate or toggle a desired valve state in order to, for example, change the aerosol concentration or composition, or change the resistance to draw of the device according to the user's preference.

The possible states of the valve 7 can also be controlled electrically within the control circuit 5 or mechanically. For example, the device may comprise a means for manually adjusting the valve, such as a screw, in order to change a position of the valve 7 or a force acting on the valve 7. This can be used to change the maximum or minimum restriction of air flow by the valve. Additionally, such adjustments can be used to change a threshold pressure or threshold air flow rate at which the valve changes state.

The aerosol generation device may be configured to control other components in tandem with the valve 7. For example, the control circuit 5 may be configured to control the aerosol generation chamber 3 based on the state of the valve 7. In one specific example, a user inhales strongly, indicating that they wish to inhale a large quantity of aerosol. In response, the valve 7 moves to a state in which the air flow is less restricted so that the user can inhale more easily. At the same time the aerosol generation chamber 3 is controlled to generate aerosol faster, for example by increasing the temperature of a heating element. The combined effect of opening the valve 7 and increasing aerosol generation means that the user is more easily able to inhale a larger quantity of aerosol.

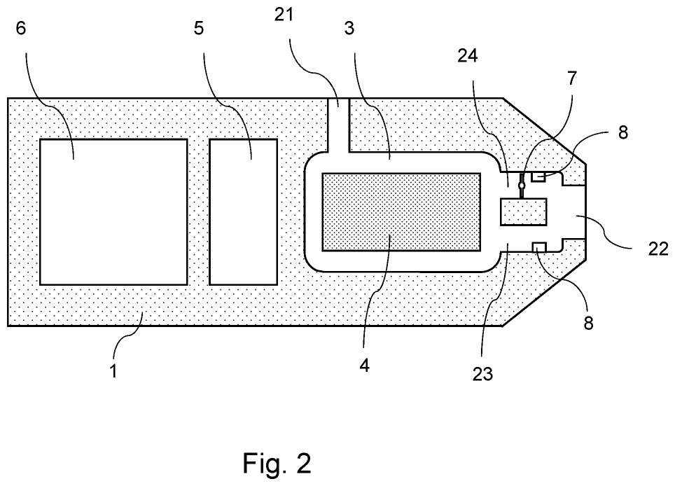

FIG. 2 is a schematic illustration of an aerosol generation device according to a second embodiment. The second embodiment is largely similar to the first embodiment, but differs in that the aerosol generation device comprises a sensor 8 in each of the channels 23 and 24.

The sensor 8 is configured to measure a characteristic of air flow in the air flow channel. For example, each sensor may be a pressure sensor or a flow rate sensor. The sensors 8 may be used to measure a difference in pressure or flow rate between the two channels 23 and 24.

Alternatively, an air flow characteristic in the air flow channel may be measured using the valve 7 itself. For example, as described above, the valve may change its state passively in response to pressure or air flow in the air flow channel. Even when the valve has not changed its state, the valve will experience a forces or torque as air flows past or when a pressure difference exists. The control circuit may detect such changes in the state of the valve 7 or forces on the valve, for example using feedback through an actuator or by using another means such as a light gate. The control circuit can then be configured to infer an air flow characteristic from the state of the valve.

Such measurement of an air flow characteristic may be used by the control circuit 5 as a trigger for controlling a state of the valve 7, as discussed above in any of the examples implementations of the first embodiment.

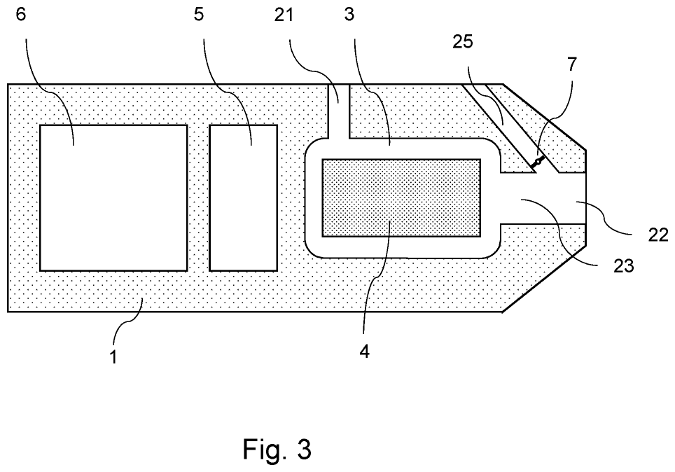

FIG. 3 is a schematic illustration of an aerosol generation device according to a third embodiment. The third embodiment is largely similar to the first embodiment, but differs in the channel 24 is omitted and the air flow channel comprises an alternative channel 25 arranged for drawing air through the aerosol generation device to the outlet 22 without passing through the aerosol generation chamber 3.

In this configuration, when the user inhales at the outlet 22 they can obtain a mixture of air drawn through the aerosol generation chamber 3, which includes the aerosol, and air drawn from outside the aerosol generation device. The air drawn through the channel 25 has the effect of diluting the aerosol in the air inhaled by the user.

In this embodiment, the valve 7 is used to control dilution of the inhaled aerosol, but otherwise operates and is controlled similarly to any of the examples given for the first or second embodiment.

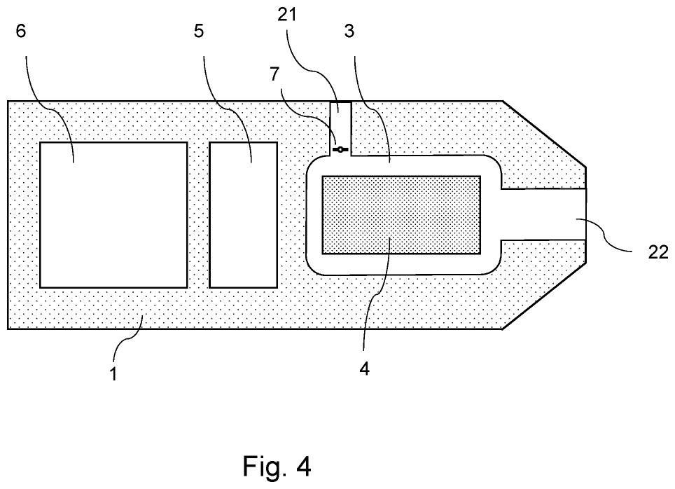

FIG. 4 is a schematic illustration of an aerosol generation device according to a fourth embodiment. The fourth embodiment is largely similar to the first embodiment, but instead of controlling air flow using a valve downstream of the aerosol generation chamber 3, the valve 7 is located in the air flow channel upstream of the aerosol generation chamber (i.e. between the aerosol generation chamber 3 and the inlet 21). Correspondingly, in this embodiment, the channels 23 and 24 are omitted and the aerosol generation chamber 3 is connected to the outlet 22.

In this embodiment, the valve 7 is configured to restrict air flow from the inlet 21 to the aerosol generation chamber 3. A valve arranged to restrict air flow in this position can, for example, decrease the minimum pressure in the aerosol generation chamber 3 when a user inhales at the outlet 22. This may be advantageous to increase evaporation of a constituent of the aerosol from the aerosol substrate 4.

The valve 7 may otherwise operate and be controlled similarly to any of the examples given for the previous embodiments.

1. An aerosol generation device comprising:
an aerosol generation chamber configured to generate an aerosol;
an air flow channel arranged for drawing air through the aerosol generation chamber and for drawing aerosol from the aerosol generation chamber to an outlet; and
a valve arranged in the air flow channel and configured to move between a first state and a second state, wherein air flow in the air flow channel in the first state is restricted more than air flow in the air flow channel in the second state,
wherein the valve is configured to move from the first state to the second state when a pressure difference across the valve exceeds a predetermined threshold.

2. The aerosol generation device according to claim 1, wherein the valve is a butterfly valve.

3. The aerosol generation device according to claim 1, wherein the air flow channel comprises a first channel in parallel with a second channel, and the valve is arranged in the second channel.

4. The aerosol generation device according to claim 3, wherein the first channel and the second channel are each connected to the aerosol generation chamber.

5. The aerosol generation device according to claim 3, wherein the first channel is connected to the aerosol generation chamber and the second channel is arranged for drawing air through the aerosol generation device without passing through the aerosol generation chamber.

6. The aerosol generation device according to claim 1, wherein the valve is arranged in the air flow channel upstream from the aerosol generation chamber.

7. The aerosol generation device according to claim 1, wherein the valve is arranged in the air flow channel downstream from the aerosol generation chamber.

8. The aerosol generation device according to claim 1, further comprising a control circuit configured to detect or control a state of the valve.

9. The aerosol generation device according to claim 8, wherein the control circuit comprises a pressure sensor or flow rate sensor arranged in the air flow channel.

10. The aerosol generation device according to claim 8, wherein the control circuit is configured to control the aerosol generation chamber based on the state of the valve.

11. The aerosol generation device according to claim 8, wherein the control circuit is configured to control the valve based on a characteristic of air flow in the air flow channel.

12. The aerosol generation device according to claim 8, wherein the control circuit is configured to control the valve based on an aerosol generation parameter.

13. The aerosol generation device according to claim 1, wherein the valve is configured to be controlled by a user.
